(19) United States
(12) Patent Application Publication
(10) Pub. No.: US 2021/0208693 A1
(43) Pub. Date: Jul. 8, 2021

(54) Method, Device and System to Deliver and Use Stress Management Psychological Tools with Near Frequency Communication (NFC) Technology

(71) Applicant: Root Success Solutions LLC, New London, CT (US)

(72) Inventor: Melissa M. Root, New London, CT (US)

(73) Assignee: Root Success Solutions LLC, New London, CT (US)

(21) Appl. No.: 17/141,544

(22) Filed: Jan. 5, 2021

Related U.S. Application Data
(60) Provisional application No. 62/958,026, filed on Jan. 7, 2020.

(51) Int. Cl. G06F 3/02; G06F 3/023; G06F 16/9538; H04W 4/90

(52) U.S. Cl. CPC G06F 3/0227; G06F 3/023; H04W 4/90; G06F 16/9538; G06F 3/0202

(57) ABSTRACT

Disclosed herein is a method that comprises obtaining a keyfob or other component having near frequency communication (NFC) technology contained therein, obtaining a computer, positioning the keyfob within 0.3 cm to 6 cm of the computer for at least ¼ second in order to access a first webpage on the computer based on instructions sent through the keyfob, and generating an output from the computer via the first webpage in response to instructions received through the keyfob, wherein the output comprises at least one audio, video and static visual output configured to induce a relaxation response in a user. A corresponding keyfob or other component, and a system, also are disclosed.

### BACKGROUND

Psychological tools may be used to address anxiety, depression, and stress management in humans. These psychological tools, when practiced, can allow humans to improve their reactions to stressful situations. Given the significant escalation of suicide in the United States since 2006, efforts to share effective mental health practices is of paramount importance. However, psychological tools for stress management are historically delivered during in-person sessions with a therapist. Some can also be found on the internet; however, the quality of these online therapies is variable. Apps that contain psychological tools exist but use of them is limited to those who seek, download, pay for, and open them. Thus, psychological tools for stress management are typically utilized by people seeing a therapist, which can be expensive and may have a stigma associated with it, or those with prior knowledge who actively seek them through apps or online searches. There are many others who can benefit from learning positive methods to manage their stress, particularly before they escalate into a mental health crisis. Getting effective psychological tools to them in a simple way is a crucial step to address the rapidly escalating mental health challenges evidenced in society.

It would be useful to develop improved methods and systems for providing individuals with stress management tools.

### SUMMARY

One embodiment disclosed herein is method, comprising obtaining a keyfob having near-frequency communication technology contained therein, obtaining a computer, positioning the keyfob within 0.3 cm to 6 cm of the computer for at least ¼ second in order to access a first webpage on the computer based on instructions sent through the keyfob, and generating an output from the computer via the first webpage in response to instructions received through the keyfob, wherein the output comprises at least one audio, video and/or static visual output configured to induce a relaxation response in a user.

Another embodiment is a keyfob comprising a housing, a near frequency communication device containing within the housing, the device being configured to communicate with a computer when positioned at a distance between 0.3 cm and 6 cm away from the computer for at least ¼ second, the device instructing the computer to automatically access a webpage providing at least one link to at least one audio, video and/or static visual output configured to induce a relaxation response in a user.

Yet another embodiment is a system comprising the above-described keyfob and a computer. In embodiments, the keyfob is physiological sensor-free. In some cases, the computer is physiological sensor-free.

A further embodiment disclosed herein is method, comprising obtaining a component having near-frequency communication technology contained therein, obtaining a computer, positioning the component within 0.3 cm to 6 cm of the computer for at least ¼ second in order to access a first webpage on the computer based on instructions sent through the component, and generating an output from the computer via the first webpage in response to instructions received through the component, wherein the output comprises at least one audio, video and/or static visual output configured to induce a relaxation response in a user. In some cases, the component is a keyfob or a placard.

Another embodiment is a component comprising a housing, a near frequency communication device containing within the housing, the device being configured to communicate with a computer when positioned at a distance between 0.3 cm and 6 cm away from the computer for at least ¼ second, the device instructing the computer to automatically access a webpage providing at least one link to at least one audio, video and/or static visual output configured to induce a relaxation response in a user. In some cases, the component is a keyfob or a placard.

Yet another embodiment is a system comprising the above-described component and a computer. In embodiments, the component is physiological sensor-free and is not associated with a sensor. In some cases, the computer is physiological sensor-free.

### BRIEF DESCRIPTION OF THE DRAWINGS

FIG. 1 illustrates a system for delivering a psychological tool in accordance with a first embodiment.

FIG. 2 is a flow chart illustrating a method to access a psychological tool.

FIG. 3 is a flow chart showing a method of reminding an individual to access and use a psychological tool.

FIG. 4 is a photo showing a front view of one embodiment of a keyfob.

FIG. 5 is a back view of the keyfob shown in FIG. 4.

FIG. 6 is a side view of the keyfob shown in FIG. 4.

FIG. 7 shows a screen shot displaying a Notifications message on a smartphone associated with a keyfob of the first embodiment.

FIG. 8 is a screen shot of an example of a webpage displayed on a smartphone that is linked to the Notifications message shown in FIG. 7.

FIG. 9 is a screen shot of the webpage shown in FIG. 8 after a user has scrolled down the page.

FIG. 10 is a screen shot of the home page after the user has further scrolled down the page.

FIG. 11 is a front view of a second embodiment, comprising a placard.

FIG. 12 is a side view of the second embodiment.

FIG. 13 is a back view of the second embodiment.

FIG. 14 is front view of a third embodiment, comprising a multi-function placard.

### DETAILED DESCRIPTION

The embodiments disclosed herein promote personal wellness by using: (1) near frequency communication (NFC) technology pre-programmed to connect to a website containing psychological tool(s) as a method to deliver stress management techniques and (2) NFC-embedded device(s) as a means to physically remind a person to use the psychological tools learned in (1). The NFC technology is programmed to connect a user's smartphone (or other NFC-enabled device) directly to a webpage that contains psychological tool(s) for stress management, including progressive muscle relaxation recording(s). The NFC technology is embedded in a device, such as a keychain, bracelet, etc., and the NFC-embedded device itself then serves as a physical object to remind the person to actively use learned psychological tools.

### Definitions:

As used herein, “keyfob” refers to a near frequency communication (NFC) embedded device carried by a user on a key ring, in a pocket, on an elongated device such as a chain or string that can be worn as a lanyard, necklace or bracelet, on a pocketbook or wallet, etc. that is configured to cause a nearby NCF-enabled computer to link to a webpage from which a user can play an audio, video and/or static visual output.

As used herein, the term “computer” refers to an NFC-enabled computerized device such as a smartphone, personal computer (PC), laptop computer, notebook computer, tablet computer, personal digital assistant (PDA), mainframe computer, MP3 player or other portable electronic device, or other appropriate computer, such as home appliances, televisions, stereos, audio-visual equipment, including recording devices, security devices, printers, medical devices, and other devices having an embedded processor, or any machine having or locally associated with data storage capability.

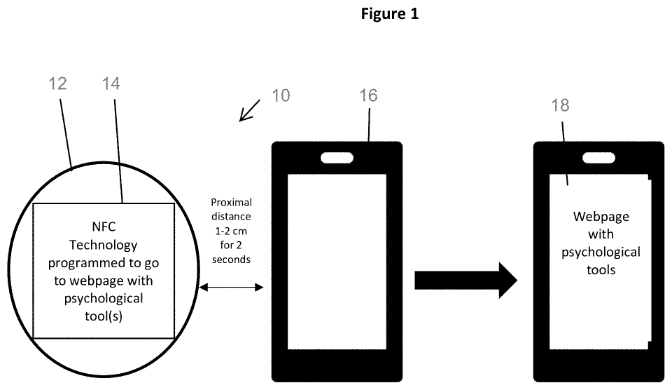

FIG. 1 describes how an NFC-enabled device and NFC technology interact to access a webpage having links to psychological tools. The method is generally designated as 10. A user positions a keyfob 12 that contains NFC technology, shown at 14, near an NFC-enabled computer 16, such as a mobile phone, with which it is configured to interact. The keyfob has a specific URL coded into the NFC tag. The computer 16 automatically links to a webpage 18 that provides links to psychological tools. The tools can be delivered using at least one of audio output, video output, a static image output of a picture, and a static image output of text. In embodiments, the keyfob 12 is positioned at a distance of about 0.3 cm to about 6 cm, or about 0.5 cm to about 4 cm, or about 1 cm to about 3 cm away from the mobile device 16 in order to automatically connect the mobile device 16 to the webpage 18. In embodiments, the keyfob 12 is held that this location proximate the mobile device 16 for a period of about 0.5 seconds to about 5 seconds, or about 1 second to about 4 seconds, or about 1.5 to about 3 seconds.

In some cases, the user activates a psychological tool by pressing a “play” or “start” button on a touch screen displayed on the computer. In other cases, the webpage link opens automatically to display an image or text, or to commence audio or video output.

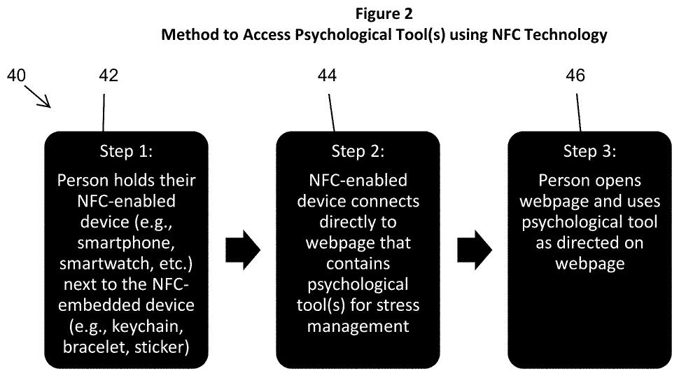

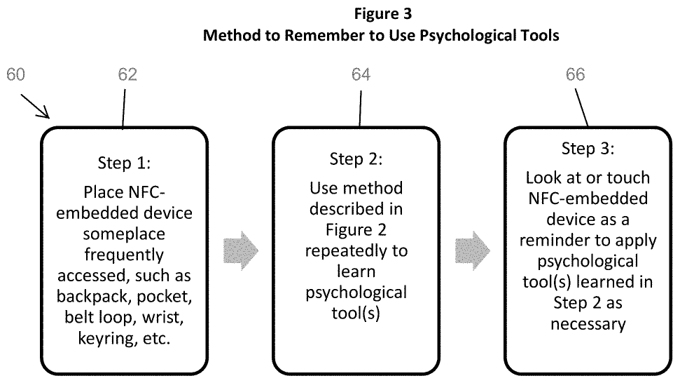

FIG. 2 illustrates a method to Access Psychological Tool(s) using NFC Technology. The overall method is designated as 40. First, the user holds their NFC-enabled device next to the NFC-embedded device at 42. Next, at 44, the keyfob 12 connects to a webpage that has tools for stress management. Subsequently, at 46, the user opens the webpage in order to use the psychological tools.

FIG. 3 illustrates a method to remember to use psychological tools. The overall method is designated as 60. First, at 62, the user places their keyfob 12 in a convenient place, such as on a keyring, backpack, bracelet, etc. Next, at 64, when looking at the keyfob 12, the user is aware that they are able to apply the psychological tools when needed. In the future, the user is reminded at 66 to use the psychological tools on an as-needed basis when they see the keyfob 12.

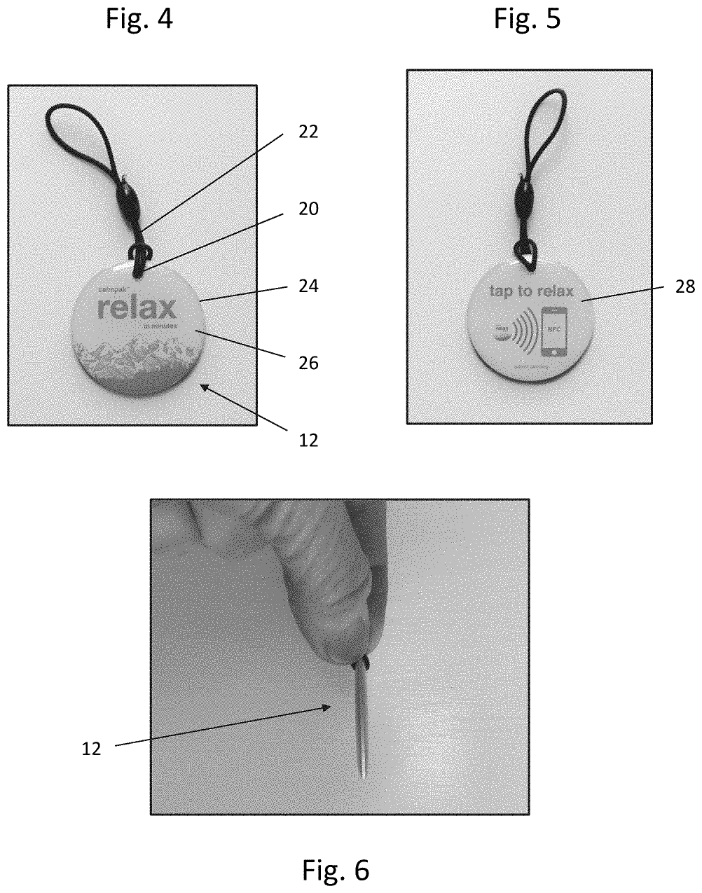

FIGS. 4-6 show various views of one embodiment of the keyfob 12. While the version shown in the figures is circular, the keyfob can have another suitable shape to be held comfortably in a user's hand, such as an oval, rectangle, hexagon, heart, cloud, rainbow, moon, etc. In embodiments, the keyfob has an aperture 20 though which an elongated member 22 can be run to connect the keyfob to another component, such as a backpack or pocketbook, is sized to be held in a user's hand or carried by the user on a key chain, necklace, bracelet, etc. In embodiments, the keyfob has a length or diameter in the range of about 0.5 cm to about 6 cm, or about 1 cm to about 5 cm, or about 2 cm to about 4 cm. The width of non-circular versions of the device is within similar ranges. In embodiments, the keyfob has a thickness in the range of about 1 mm to about 15 mm, or about 1 mm to about 10 mm, or about 1 bout 5 mm. The NFC-enabling component 14 (schematically shown in FIG. 1) is surrounded by a housing 24 having sufficient durability to protect the NFC-enabling device 14 from damage when the keyfob is carried on the outside of a backpack or pocketbook. The housing 24 has a front surface 26 and a back surface 28. In the illustrated embodiment, the front surface 26 has the word “Relax” printed thereon and the back surface 28 has use instructions printed thereon. Other text and or images can be used in place of, or in addition to the text and images shown in the figures. The housing typically is made from a rigid thermoplastic or thermoset material.

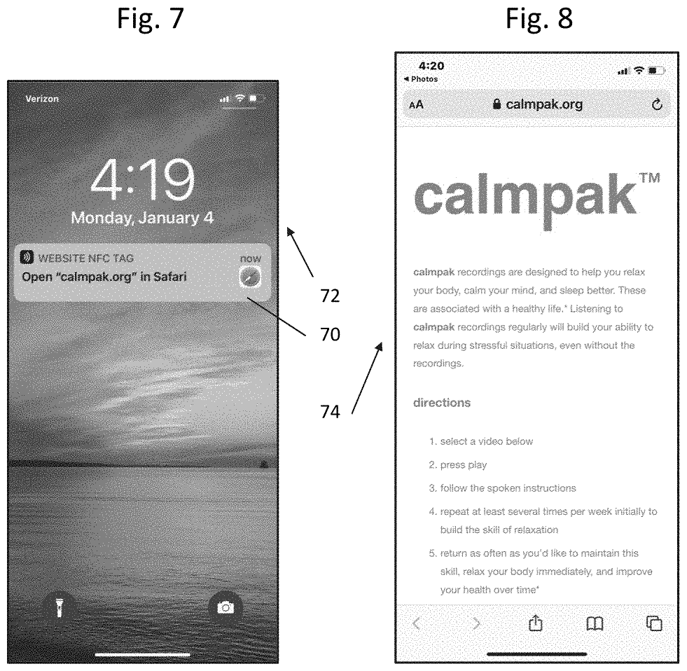

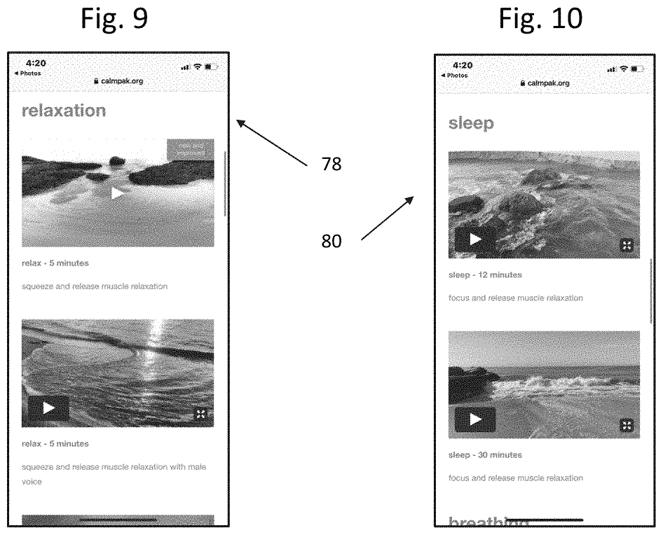

FIGS. 7-10 show screen shots of a smartphone enabled by the NFC device. When the smartphone is held near the keyfob 12, a notification, shown at 70, appears on a screen 72. After the user touches the notification, a screen shot shown at 74 appears, providing directions for a user on a home screen for the linked webpage. The user can scroll down the home page to access audio and/or video recording used to relax, shown at 78, and can scroll further down to access audio and/or video recording used to go to sleep, shown at 80.

In some cases, the keyfob 12 is configured to be accessed by a single smartphone or other NFC-enabled computer. In other cases, the keyfob is configured to be accessed by multiple smartphones or other NFC-enabled computers.

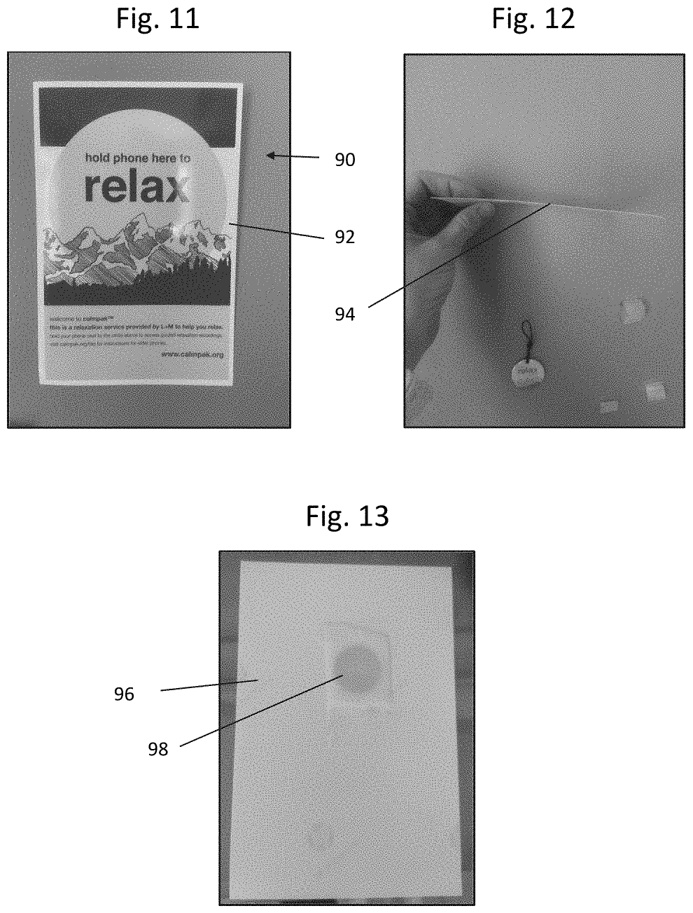

FIGS. 11-13 show a second embodiment, which is a placard that, in most cases, is configured to be accessed by multiple NFC-enabled computers. This embodiment can be used by a group of persons, such as in a hospital or school. The placard has a front surface 92 with instructions thereon, a thin side edge 94, and a rear surface 96 with an NFC tag mounted thereon, they can access the website containing psychological tools. When a particular user brings their smartphone or other computer in close proximity to the placard 90, they can access the psychological tools. The placard can be mounted on a wall, hung on a doorknob, or positioned in another suitable manner.

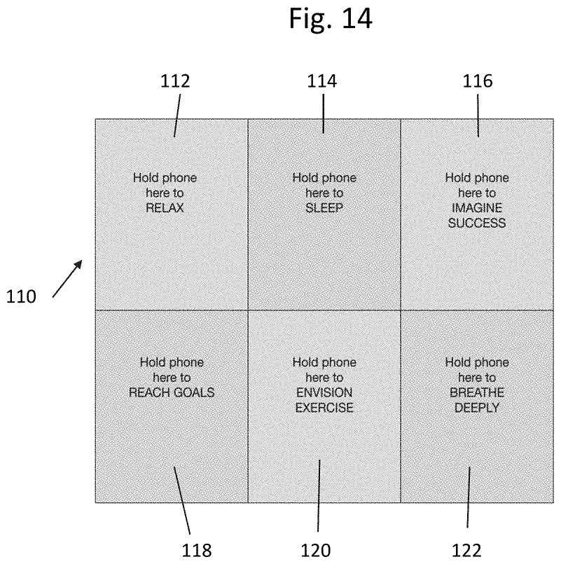

FIG. 14 shows a third embodiment in which 6 different NFC tags are mounted to different parts of a single placard 110. The user places their smartphone near rectangle 112 to access relaxation tools, or places their smartphone near rectangle 114 to access sleep-inducing tools. If a user wishes to access tools to imagine success, they place their smartphone near rectangle 116. Access to tools for reaching goals can be reached by the NFC tag in rectangle 118. The user can access tools to envision exercise at square 120 and tools for deep breathing at rectangle 122.

In some embodiments, two NFC tags can be disposed one on top of the other with a sheet of metal therebetween. The two NFC tags can provide links to two different webpages. The user can place their smartphone near one surface or the other (i.e. the front surface or the back surface of the placard or keyfob) depending upon which psychological tool they would like to access.

In embodiments, the webpage is configured for use by multiple users. The same webpage is linkable to every user with no differentiation. This can be advantageous because some individuals do not wish to use online health apps for fear identifiable data collection. Using mental health tools in some cases is associated with stigma. The keyfob eliminates that stigma by having completely anonymous use of evidence-based tools.

In other cases, the webpage is personalized to an individual user and is linkable to only the individual user. In embodiments, a therapist can periodically modify the output associated with the linked webpage.

In certain cases, the keyfob is configured to access a second webpage after accessing the first webpage a predetermined number of times. In some cases, the keyfob is configured to sequentially access multiple webpages in sequence when used multiple times.

In embodiments, the keyfob does not contain a physiological sensor, and therefore the keyfob is not activated based on a physiological characteristic of a user such as heart rate, breathing rate, etc. In embodiments, the keyfob is part of a system in which the computer does not contain a physiological sensor, and therefore the webpage is not accessed based on a physiological characteristic of a user, such as heart rate, breathing rate, etc.

In embodiments, access to the linked pages can be password protected, allowing for use of personalized programs. In embodiments, the computer is configured for voice activation of the webpage link, and therefore the user can instruction the computer to generate an output using audio instructions. In embodiments, the computer is configured to recognize the voice of an individual user.

Most importantly, the NFC-embedded keyfob can be readily handed out by organizations, corporations, schools, and individuals, thus increasing the distribution of psychological tools. The connection to the webpage with psychological tools is immediate, cost of the NFC device is minimal, need for a therapist for in-person implementation is eliminated, and prior knowledge of psychological tools is unnecessary. The methods of using NFC technology to deliver psychological tools via a webpage and using NFC-embedded devices to remind users to apply the tools learned are the inventions claimed herein.

A number of alternatives, modifications, variations, or improvements therein may be subsequently made by those skilled in the art, which are also intended to be encompassed by the following claims.

What is claimed is:

1. A method, comprising:
obtaining a keyfob having near frequency communication technology contained therein, obtaining a computer, position
ing the keyfob within 0.3 cm to 6 cm of the computer for at least ¼ second in order to access a first webpage on the computer based on instructions sent through the keyfob, and generati
ng an output from the computer via the first webpage in response to instructions received through the keyfob, wherein the output comprises at least one of an audio output, a video output and a static visual output configured to induce a relaxation response in a user.

2. The method of claim 1, wherein the keyfob is dimensioned to be capable of being held in the hand of the user.

3. The method of claim 1, wherein the first webpage is simultaneously accessible by multiple users.

4. The method of claim 1, wherein the first webpage is personalized to an individual user.

5. The method of claim 4, wherein the first webpage is linkable to only the individual user.

6. The method of claim 1, further comprising periodically modifying the output associated with the first webpage.

7. The method of claim 1, wherein the keyfob is configured to access a second webpage after accessing the first webpage a predetermined number of times.

8. The method of claim 1, wherein the keyfob is configured to sequentially access multiple webpages in sequence when used multiple times.

9. The method of claim 1, wherein a user can select an output type using a button on the computer.

10. The method of claim 1, wherein the keyfob contains at least two NFC tags and a user can select a tag associated with a particular output type by selecting a certain contact location on the keyfob.

11. The method of claim 1, wherein the keyfob is configured to remind user to access the first webpage.

12. A keyfob, comprising:
a housing, a near f
ency communication device containing within the housing, the device being configured to communicate with a computer when positioned at a distance between 0.3 cm and 6 cm away from the computer for at least ¼ second, the device instructing the computer to automatically access a webpage providing at least one link to at least one of an audio output, video output and static visual output configured to induce a relaxation response in a user.

13. The keyfob of claim 12, wherein the housing is dimensioned to be capable of being held in the hand of a user.

14. The keyfob of claim 12, wherein the housing has an outer surface having graphics formed thereon.

15. The keyfob of claim 14, wherein at least one of a shape of the housing and the graphics is configured to remind a user to use the keyfob.

16. The keyfob of claim 14, wherein the graphics include user instructions.

17. The keyfob of claim 14, wherein the keyfob is physiological sensor-free.

18. A system comprising the keyfob of claim 12 and a computer.

19. The system of claim 16, wherein the computer is a smartphone.

20. The system of claim 18 wherein both the keyfob and computer are physiological-sensor-free.
